(19) United States
(12) Patent Application Publication
(10) Pub. No.: US 2025/0332080 A1
(43) Pub. Date: Oct. 30, 2025

(54) Skin Cell Energy Booster Composition

(71) Applicant: CarboCode S.A., Cantanhede (PT)

(72) Inventors: Cornelia PYKO, Cantanhede (PT); Feryel ADAM, Cantanhede (PT); Bruce MCCONNELL, Cantanhede (PT)

(21) Appl. No.: 18/860,982

(22) PCT Filed: Apr. 28, 2023
(86) PCT No.: PCT/IB2023/054420

§ 371 Date: Oct. 28, 2024

(30) Foreign Application Priority Data
Apr. 29, 2022 (WO) PCT/PT2022/050018
Aug. 5, 2022 (PT) 118148

(51) Int. Cl. A61K 8/41; A61Q 19/02; A61Q 19/08

(52) U.S. Cl. CPC A61K 8/41; A61Q 19/02; A61Q 19/08; A61K 2800/522

(57) ABSTRACT

The present invention relates to a cosmetic use of a composition for boosting skin cell energy metabolism in an individual, wherein the composition comprises a compound of formula I, wherein the carbon-carbon bond noted Formula II is a double or a single bond, R¹ is an alkyl chain having 10-20 carbon atoms, R² is hydrogen or a substituted or unsubstituted acyl having 16-34 carbon atoms, R³ is hydrogen when the carbon-carbon bond noted is a double or a single bond, or R³ is —OH when the carbon-carbon bond noted is a single bond, R⁴ is hydrogen when the carbon-carbon bond noted is a double or a single bond, or R⁴ is hydrogen or —OH when the carbon-carbon bond noted is a double bond, X is hydrogen, a glucosyl moiety or a lactosyl moiety.

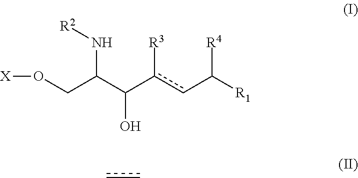

### FIELD OF THE INVENTION

The present invention relates to a use of a composition for boosting cell energy metabolism in skin cells of an individual, and to compositions for boosting skin cell energy metabolism.

### BACKGROUND OF THE INVENTION

The skin is the outer sheath of the body, which interfaces with the external environment. It is a complex structure made of several tissue layers, each with a distinct cellular composition. It has crucial functions like sensation, heat insulation and prevention of water loss, and acts as a physical barrier to pathogens. The upper layer of the skin is the epidermis, a stratified structure interspersed with hair follicles. The outer epidermal surface is made of cornified keratinocytes or corneocytes that form a dense cytoskeletal network of keratin filaments. The basal layer harbours the epidermal stem and progenitor cells. The dermis lies underneath the epidermis and consists of dermal fibroblasts that produce collagen and elastin that form the extracellular matrix (ECM), as well as the melanocytes that produce the photo-protective pigment melanin. If repair mechanisms cannot keep pace, detrimental changes in skin structure can occur, affecting the protective role of the skin against physicochemical and biological attacks, as well as its thermoregulatory, sensory, immunological and hormonal functions. Both intrinsic and environmental factors affect the epidermal and dermal layers of the skin. Intrinsic aging processes are the result of chronological, inevitable senescence of the skin cells which varies depending on ethnicity, hormones and the anatomical region of the affected skin. Extrinsic skin aging is a result of all the external factors including lifestyle, smoking, UV exposure and the environment, which have a cumulative effect over time.

The skin is a high turnover organ, with a continuously regenerating epidermis. The epidermal progenitors are thus highly proliferative and metabolically active, for which much energy is required. The energy requirements are met by mitochondrial respiration, an adenosine triphosphate (ATP) generating process driven by a series of protein complexes collectively known as electron transport chain (ETC) located on the inner membrane of the mitochondria. The mammalian mitochondrial ETC is composed of transmembrane protein complex I (NADH dehydrogenase), complex II (succinate dehydrogenase), complex III (ubiquinol-cytochrome C oxidoreductase) and complex IV (cytochrome C oxidase) and the freely mobile electron transporters ubiquinone and cytochrome C. Complex I is the first enzyme of the respiratory chain and plays a central role in energy metabolism. It oxidizes NADH, which is generated through the Krebs cycle in the mitochondrial matrix, and uses the two electrons to reduce ubiquinone to ubiquinol. Ubiquinol is reoxidized by the cytochrome be 1 complex and transfers electrons to reduce molecular oxygen to water at complex IV. The redox energy released during this process is used to transfer protons from the mitochondrial matrix to the periplasmic space that generates proton-motive force across the inner mitochondrial membrane at complex I, III, and IV.

The complexes must be assembled into a specifically configured supercomplex to function properly, and the assembled components together with ATP synthase become the basis of ATP production during oxidative phosphorylation. Natural by-products of respiration include reactive oxygen species (ROS), which play an important role in cell proliferation, hypoxia adaptation and cell fate determination, however excessive ROS can disrupt macromolecular and cellular structures if not quenched by the antioxidant system. This oxidative damage caused by mitochondrial ROS production has been established as an important molecular basis of aging (Sreedhar et al., Cell Death and Disease (2020)11:444). Complexes I and III are generally considered the main sites of ROS production where electron leaks and single electrons react with oxygen, producing superoxide anion. Complex I is involved in a variety of cellular functions ranging from apoptosis and necrosis to cell proliferation. Complex I appears to be the most vulnerable site to oxidative stress.

Intrinsic aging process in cells and skin can be related to the loss of proper function of the skin. Losses of proper function of the skin can lead to increased oxidative damage, increased inflammation, dry skin, loss of skin firmness, increased skin unevenness, and increased fine lines and wrinkles. Factors that cause extrinsic aging can include exposure to ultraviolet (UV) rays, irritants, and pollutants, such as fine particles suspended in the air. Chronic UV exposure incudes mitochondrial DNA damage and oxidative stress in the skin cells, which can progress to photo-aging. UVB mainly acts on epidermal keratinocytes and melanocytes, while UVA can penetrate more deeply into the dermis. A cardinal marker of photo-aging is large-scale mitochondrial DNA (mtDNA) deletion. Phenotypic manifestations include loss of elasticity, wrinkles and abnormal pigmentation.

With studies increasingly supporting the close association between mitochondria and skin appearance and health, its targeting in the skin, e.g. via an ATP production boost, has gained attention from clinicians and aestheticians alike. Activating the respiratory chain and therefore ATP generation keeps the metabolism high and provides energy for repair mechanisms, cells stay healthy & functional resulting in a healthy skin with a radiant complexion. Furthermore, an activated metabolism supports skin regeneration and helps to fight against signs of ageing.

There remains a need for treatments that efficiently address the above undesired changes of the skin in a comprehensive manner and help keeping a radiant and healthy skin appearance and function.

### SUMMARY OF THE INVENTION

The present inventors have surprisingly found certain compounds that are capable of boosting cell energy metabolism in skin cells. Accordingly, this invention relates to a use of a composition for boosting cell energy metabolism in skin cells of an individual, and to compositions for boosting skin cell energy metabolism.

Specifically, the present invention comprises the following aspects:
(1) Cosmetic use of a composition for boosting skin cell energy metabolism in an individual, wherein the composition comprises one or more compounds of formula I

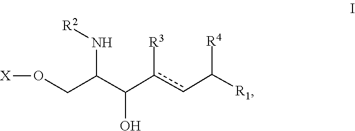

wherein
the carbon-carbon bond noted [symbol] is a double or a single bond,
R¹ is an alkyl chain having 10-20 carbon atoms,
R² is hydrogen or a substituted or unsubstituted acyl having 16-34 carbon atoms,
R³ is hydrogen when the carbon-carbon bond noted [symbol] is a double or a single bond, or
R³ is —OH when the carbon-carbon bond noted [symbol] is a single bond,
R⁴ is hydrogen when the carbon-carbon bond noted [symbol] is a double or a single bond, or
R⁴ is hydrogen or —OH when the carbon-carbon bond noted [symbol] is a double bond,
X is hydrogen, a glucosyl moiety or a lactosyl moiety.
(2) Method for boosting skin cell energy metabolism, comprising topically administering a composition comprising a compound of formula I onto skin of an individual.
Beneficial effects of a cosmetic treatment with a topical composition comprising one or more compounds of formula I include the following
   Improving skin complexion;
   Improving skin radiance;
   Improving skin tone;
   Skin soothing;
   Preventing or reducing skin ageing, such as inflamm-aging or photo-aging;
   Wrinkle smoothing;
   Supporting skin regeneration;
   Reducing signs of skin inflammation such as itching, redness or swelling; and/or
   Reducing signs of skin fatigue.
(3) Pharmaceutical topical composition comprising a compound of formula I for use in treating a skin disease in an individual.

Various non-limiting embodiments relating to the above aspects of the invention are described throughout specification of the invention and illustrated by non-limiting examples.

### FIGURES

FIGS. 1 to 3 show heatmaps using a log 2 scale with up- or downregulation of respective proteins upon treatment with 0.01% (01), 0.03% (03) and 0.15% (15) of respective ceramide in vehicle as compared to vehicle without ceramide (VEH) (FIG. 1: GlcCerNS, FIG. 2: LacCerNS, FIG. 3: CerNG); detailed experimental setup explained in Example 1.

FIG. 4 shows activity of complex I in skin explants (expressed in U/g protein) of tissue control on day 0 (TO), and of non-treated control (T), treated with basis emulsion (E) and treated with basis emulsion with GlcCer (P), respectively, on day 6.

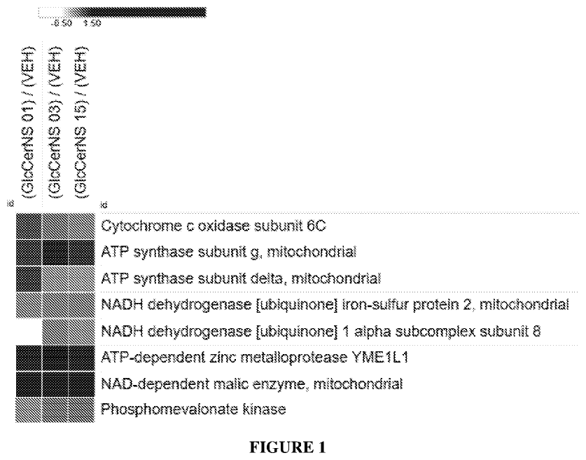

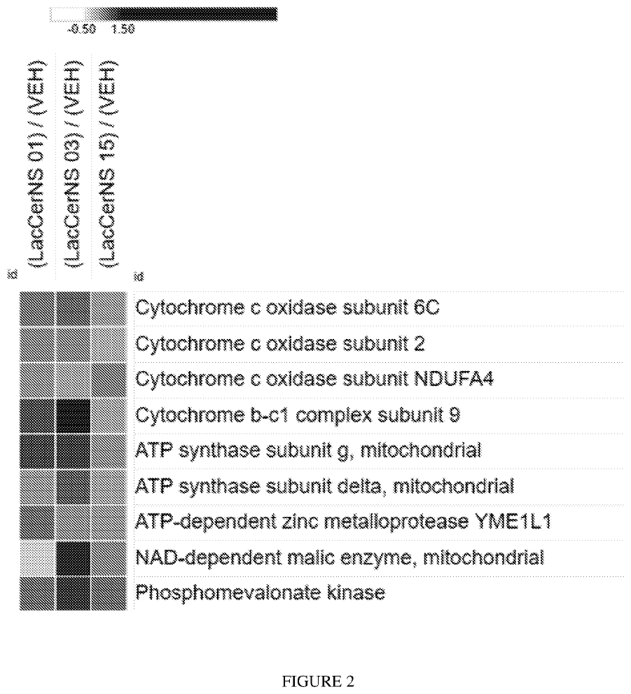

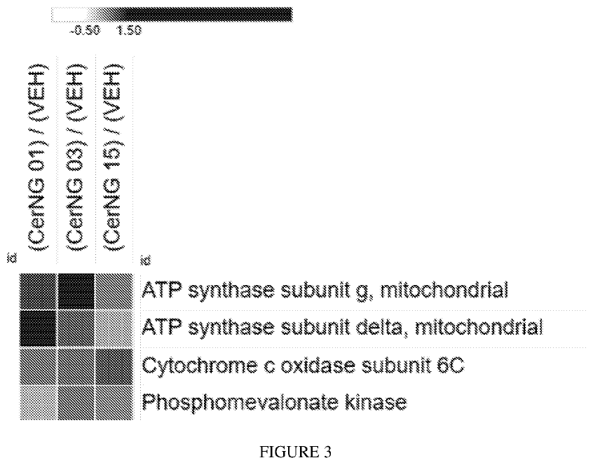

### DETAILED DESCRIPTION OF THE INVENTION

It has now been surprisingly found that compounds of formula I boost cell energy metabolism in skin cells. Boosting energy of the skin cells as used herein refers to an improvement of mitochondrial functions, including ATP-production, and may also be referred to as skin vitalisation or skin energizing.

In this specification, the following terms have the following meanings:

“Topical composition” includes compositions suitable for topical application on keratinous tissue, especially on skin. Such compositions are typically dermatologically acceptable in that they do not have undue toxicity, incompatibility, instability, allergic response, and the like, when applied to skin. Topical compositions typically comprise a topically acceptable carrier. Topical skin care compositions of this invention can have a selected viscosity to avoid significant dripping or pooling after application to skin. Accordingly, a topical composition of the invention comprising one or more compounds of formula I, would typically comprise one or more other compounds that are useful for formulating these compositions. Typically, such “formulating” compounds do not possess the biological activity of the compounds of the invention.

“Keratinous tissue” includes keratin-containing layers disposed as the outermost protective covering of mammals and includes, but is not limited to, lips, skin, scalp, hair and nails.

The terms “about” or “approximately” are defined as being close to the value or range following these terms, as understood by one of ordinary skill in the art, and include a deviation of up to 10% of the values or ranges that follow.

The term “reducing” or any variation of the term such as “reduction” includes any measurable decrease to achieve a desired effect.

The terms “promoting”, “enhancing”, “improving” or “increasing” or any variation of these terms include any measurable increase to achieve a desired effect.

“Effective amount” means an amount sufficient to render a desired treatment or management outcome in a human. An effective amount can be administered in one or more doses to achieve the desired treatment or management outcome.

“Preventive treatment”, “preventing” or “prevention” means treatment given or action taken to diminish the risk of onset or recurrence of a cosmetic or medical condition, or of an effect related to an impairment in the visual appearance of a skin or of a skin disease.

“Primary prevention” means prevention of the initial onset of a cosmetic or a medical condition in an individual.

“Secondary prevention” means, in an individual who has a cosmetic or a medical condition or who has had a condition, (i) prevention of reoccurrence of the condition, (ii) increase in the duration of remission of the condition, and/or (iii) reduction in severity of symptoms of the condition.

“Treat” means to address a cosmetic or a medical condition or disease with the objective of improving or stabilising an outcome in the person being treated or addressing an underlying need. Treat therefore includes the topical management of the cosmetic or medical condition or disease by addressing dermatological needs of the person being treated. “Treating” and “treatment” have grammatically corresponding meanings.

“Therapy” means treatment given or action taken to reduce or eliminate symptoms of a disease or pathological condition.

The terms defined above and embodiments described throughout the specification are applicable to all aspects of the invention, unless specified otherwise.

In one aspect, the present invention relates to a cosmetic use of a composition for boosting cell energy metabolism in an individual, wherein the composition comprises a compound of formula I

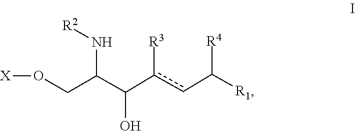

wherein
the carbon-carbon bond noted [symbol] is a double or a single bond,
R¹ is an alkyl chain having 10-20 carbon atoms,
R² is hydrogen or a substituted or unsubstituted acyl having 16-34 carbon atoms,
R³ is hydrogen when the carbon-carbon bond noted [symbol] is a double or a single bond, or
R³ is —OH when the carbon-carbon bond noted [symbol] is a single bond,
R⁴ is hydrogen when the carbon-carbon bond noted [symbol] is a double or a single bond, or
R⁴ is hydrogen or —OH when the carbon-carbon bond noted [symbol] is a double bond,
X is hydrogen, a glucosyl moiety or a lactosyl moiety.

As used herein, the term “alkyl” refers to an acyclic straight hydrocarbyl group in which the carbon atoms may be saturated or contain one or more double and/or triple bonds (so, forming for example an alkenyl or an alkynyl). Examples of “alkyl” include, but are not limited to, methyl, ethyl, n-propyl, isopropyl, isobutyl, n-butyl, sec-butyl, tert-butyl, isopentyl, n-pentyl, neo-pentyl, n-hexyl, ethenyl, propenyl, 1-butenyl, 2-butenyl, isobutenyl, 1-pentenyl, 2-pentenyl, 2-methyl-1-butenyl, 3-methyl-1-butenyl, 2-methyl-2-butenyl, 1-hexenyl, 2-hexenyl, 3-hexenyl, methylpentenyl, dimethylbutenyl, ethynyl, propynyl, 1-butynyl, 2-butynyl, pentynyl, and hexynyl. Typically, the term alkyl refers to a saturated hydrocarbyl group.

As used herein, the term “acyl” includes both saturated groups and groups having one or more double and/or triple bonds. In the context of acyls, the term “substituted” means that the carbon atoms may be substituted one or several times, preferably 1 to 3 times, with functional group(s).

In a preferred embodiment, R² of compound I is an unsubstituted acyl group. In a more preferred embodiment, R² of compound I is a saturated fatty acyl group or a fatty acyl group having one or more double bonds.

The compound of formula I may be obtained by any technique known to the skilled person, such as by a fermentation technique or by a biocatalytic process. For example, glucosylceramide may be produced starting from glucosyl fluoride, followed by coupling to an acylated sphingoid base by the use of an endoglycoceramide synthase, such as described in Rich et al., ChemComm (2011) 47:10806-10808. Sphingoid bases may be produced by a fermentation process, such as e.g. described in WO 95/12683 A1, especially phytosphingosine or a precursor thereof (claim 1 of EP 0 726 960 B1).

In some embodiments, R¹ of the compound of formula I is —C₁₂H₂₅ and/or R² is hexadecanoyl, 2-hydroxyhexadecanoyl, octadecanoyl, 2-hydroxyoctadecanoyl, eicosanoyl, 2-hydroxyeicosanoyl, lignoceroyl, 2-hydroxytetracosanoyl or 30-(linoleyloxy)-triacontanoyl, preferably octadecanoyl or lignoceroyl.

In some embodiments, R¹ of the compound of formula I is —C₁₂H₂₅ and R² is octadecanoyl.

The composition comprising the compound of formula I may be a topical composition or an oral composition. Typically, the composition comprising the compound of formula I is a topical composition.

The compound of formula I may be formulated into topical compositions suitable for application to the skin. Generally, the topical compositions of the present invention will contain from about 0,0001% to about 10% wt/wt of a compound of formula I, such as from about 0,005% to about 5% wt/wt of a compound of formula I, or from 0.01% to about 1% wt/wt of a compound of formula I. For example, the topical compositions of the present invention may contain from about 0,0001 to about 0,00025% wt/wt of a compound of formula I, from about 0,0002 to about 0,0004% wt/wt of a compound of formula I, from about 0,0003 to about 0,0005% wt/wt of a compound of formula I, from about 0,0004 to about 0,0006% wt/wt of a compound of formula I, from about 0,0005 to about 0,0008% wt/wt of a compound of formula I, from about 0,0007 to about 0,001% wt/wt of a compound of formula I, from about 0.001 to about 0,005% wt/wt of a compound of formula I, from about 0,003 to about 0,008% wt/wt of a compound of formula I, from about 0,005 to about 0.01% wt/wt of a compound of formula I, from about 0,008 to about 0.05% wt/wt of a compound of formula I, from about 0.05 to about 0.08% wt/wt of a compound of formula I, from about 0.06 to about 0.1% wt/wt of a compound of formula I, from about 0.08 to about 0.1% wt/wt of a compound of formula I, etc.

In some embodiments, the topical compositions of the present invention contain an amount of about 0.01% to 5% wt/wt of a compound of formula I, or an amount of about 0.02% wt/wt to 1% wt/wt of a compound of formula I. Examples are 0.05% wt/wt, 0.1% wt/wt or 0.2% wt/wt of a compound of formula I.

The topical composition may in some embodiments comprise more than one compound of formula I, also termed herein as blend of compounds of formula I. In such blend compositions, each compound of formula I may be present in the same amount or in different amounts. The ranges or amounts as listed above may relate to the total content of the blend in the topical composition, or to the amount of one compound in the blend in the topical composition.

The amount of the compounds of formula I in a composition of the invention may vary depending upon factors such as the age of the person, the risk and severity of any underlying condition, the environment the person is exposed to, the form of the topical composition, its use for cosmetic or medical purposes, and any other skin care compositions being administered. Appropriate doses for any particular person or indication may be determined by methods known to skin care practitioners. The dose may also vary depending upon whether the topical composition is a “leave on” or “rinse off” formulation with “rinse off” formulations generally containing lower doses. Further, the dose may vary depending on whether the topical composition is applied in an intervention treatment phase or in a maintenance phase. Generally higher doses can be applied in an initial intervention phase than in a maintenance phase.

The composition according to the present invention may have a pH in the range of about 4.5 to about 8, e.g. from about 4.5 to about 6.

In the context of the invention it has been surprisingly found that application of glucosyl ceramide (GlcCer) on keratinocytes leads to an upregulation of proteins of the mitochondrial respiratory chain. More precisely, the application of GlcCer leads to an upregulation of several mitochondrial proteins associated with generation of ATP, in particular ATP synthase subunit g (mitochondrial), of ATP synthase subunit delta (mitochondrial), of cytochrome c oxidase subunit 6C, of NADH dehydrogenase [ubiquinone]iron-sulfur protein 2 (mitochondrial), of NADH dehydrogenase [ubiquinone]1 alpha subcomplex subunit 8, of ATP-dependent zinc metalloprotease YME1L1, of NAD-dependent malic enzyme (mitochondrial) and of phosphomevalonate kinase.

In some embodiments, glucosyl ceramide is used for up-regulating ATP synthase subunit g.

In some embodiments, glucosyl ceramide is used for up-regulating ATP synthase subunit delta.

In some embodiments, glucosyl ceramide is used for up-regulating cytochrome c oxidase subunit 6C.

In some embodiments, glucosyl ceramide is used for up-regulating NADH dehydrogenase [ubiquinone]iron-sulfur protein 2.

In some embodiments, glucosyl ceramide is used for up-regulating NADH dehydrogenase [ubiquinone]1 alpha subcomplex subunit 8.

In some embodiments, glucosyl ceramide is used for up-regulating ATP-dependent zinc metalloprotease YME1L1.

In some embodiments, glucosyl ceramide is used for up-regulating NAD-dependent malic enzyme.

In some embodiments, glucosyl ceramide is used for up-regulating phosphomevalonate kinase.

In the context of the invention it has been further found that application of lactosyl ceramide on keratinocytes leads to an upregulation of ATP synthase subunit g (mitochondrial), of ATP synthase subunit delta (mitochondrial), of cytochrome c oxidase subunit 6C, of cytochrome c oxidase subunit 2, of cytochrome c oxidase subunit NDUFA4, of cytochrome b-c1 complex subunit 9, of ATP-dependent zinc metalloprotease YME1L1, of NAD-dependent malic enzyme (mitochondrial) and of phosphomevalonate kinase.

In some embodiments, lactosyl ceramide is used for up-regulating ATP synthase subunit g.

In some embodiments, lactosyl ceramide is used for up-regulating ATP synthase subunit delta.

In some embodiments, lactosyl ceramide is used for up-regulating cytochrome c oxidase subunit 6C.

In some embodiments, lactosyl ceramide is used for up-regulating cytochrome c oxidase subunit 2.

In some embodiments, lactosyl ceramide is used for up-regulating cytochrome c oxidase subunit NDUFA4.

In some embodiments, lactosyl ceramide is used for up-regulating cytochrome b-c1 complex subunit 9.

In some embodiments, lactosyl ceramide is used for up-regulating ATP-dependent zinc metalloprotease YME1L1.

In some embodiments, lactosyl ceramide is used for up-regulating NAD-dependent malic enzyme.

In some embodiments, lactosyl ceramide is used for up-regulating phosphomevalonate kinase.

In the context of the invention it has been further found that application of ceramide NG on the skin leads to an upregulation of ATP synthase subunit g (mitochondrial), of ATP synthase subunit delta (mitochondrial), of cytochrome c oxidase subunit 6C and of phosphomevalonate kinase.

In some embodiments, ceramide NG is used for up-regulating ATP synthase subunit g.

In some embodiments, ceramide NG is used for up-regulating ATP synthase subunit delta.

In some embodiments, ceramide NG is used for up-regulating cytochrome c oxidase subunit 6C.

In some embodiments, ceramide NG is used for up-regulating phosphomevalonate kinase.

NADH dehydrogenase (ubiquinone) iron-sulfur protein 2 and NADH dehydrogenase (ubiquinone) 1 alpha subcomplex subunit 8 form part of complex I of the ETC:

Cytochrome b-c1 complex subunit 9 forms part of complex III of the ETC.

Cytochrome C oxidase subunit 6C, cytochrome C oxidase subunit 2 and Cytochrome C oxidase subunit NDUFA4 form part of complex IV of the ETC.

ATP synthase subunit g and ATP synthase subunit delta form part of the ATP synthase complex.

Phosphomevalonate kinase is part of the Phosphomevalonate pathway which generates precursor for Ubiquinone. Ubiquinone is required in the respiratory chain for electron transport.

NAD-dependent mitochondrial malic enzyme catalyzes the oxidative decarboxylation of malate to pyruvate. Pyruvate is used in the tricarbon acid cycle which generates activated electrons for the respiratory chain.

ATP-dependent zinc metalloprotease ensures cell proliferation, maintains normal mitochondrial cristae morphology and complex I respiration activity, promotes antiapoptotic activity and protects mitochondria from the accumulation of oxidatively damaged membrane proteins.

Without being bound to theory, it is expected that an up-regulation of these markers lead to an increase in mitochondrial ATP production and consequently a boost in cell energy. In turn, adverse phenotypic manifestations caused by mitochondrial dysfunctions, including signs of ageing, especially inflamm-aging and photo-aging, which further impact the healthy look of the skin, including skin complexion, skin tone or skin radiance, would be prevented or reduced.

The one or more compounds in the composition according to the present invention may accordingly be especially glucosyl ceramide, lactosyl ceramide and/or ceramide NG.

Glucosyl ceramide (GlcCER) as mentioned herein is preferably ceramide NS and has an acyl chain length of Cis. Glucosyl ceramide NS with an acyl chain length of C₁₈ may be referred to herein also as GlcCNS or as GlcCNS18. The lactosyl ceramide (LacCER) as mentioned herein is preferably ceramide NS, typically with an acyl chain length of Cis. Lactosyl ceramide NS with an acyl chain length of C₁₈ may be also referred to herein as LacCNS.

Most preferably, one compound in the composition according to the present invention is GlcCNS18.

Ceramide NG, ceramide NS and ceramide NP are named according to the shorthand nomenclature developed by Motta et al., Biochim Biophys Acta., 1993, 1182:147-151 and expanded by Rabionet et al., Biochim Biophys Acta, 2014, 1841:422-434, and by Masukawa et al., Journal of Lipid Research, 2008, 49, 1466-1476. Ceramides NG may also be referred to as ceramide NdS. For each species, the number of carbons and unsaturations (if present) may be expressed in parentheses following the letters of N, A, E, and O. When not specified, the carbon chain length of the sphingoid base moiety of the ceramides mentioned herein is typically C₁₈. When not specified, the carbon chain length of the acyl moiety of the ceramides mentioned herein may be e.g. C₁₆, C₁₇, C₁₈, C₁₉, C₂₀, C₂₂ or C₂₄.

The inventive use in general relates to an individual who is a mammal. Especially, the individual is a human. In some preferred embodiments, the human individual is an adult individual, i.e. a human of 20+ years old, especially 30+ years old, such as 40+ years old or 50+ years old; in other preferred embodiments, the human individual may be a child or a teenager. The use of the inventive topical composition for a younger individual is especially useful when intended for prophylactic or preventive treatment, or when the individual is in a situation in which the visual appearance of the skin, especially facial and neck skin, is negatively affected, such as when the individual is prone to premature skin aging or suffers from a disease that affects the visual appearance of the skin.

In some embodiments, the topical composition may advantageously comprise a further compound which is known for or capable of boosting cell energy metabolism in skin cells of an individual. Examples include enzyme cofactors, such as Coenzyme Q10, vitamins, e.g. vitamin B3 (niacin or niacinamide) or vitamin C, an agent that is capable of deporting mitochondrial ATP and facilitating ATP recycling, such as creatine, an anti-oxidant, e.g. resveratrol or astaxanthin, or a combination thereof. This further compound may be preferably Coenzyme Q10, vitamin B3 or creatine.

Cosmetic use or treatment involving a composition comprising a compound of formula I is intended to improve the visual appearance of skin of the treated individual. By the wording “improve visual appearance of skin” is meant that cosmetic treatment with a composition of the invention comprising a compound of formula I, in particular resulting in one or more of the following beneficial effects on the treated skin of an individual:
Improving skin complexion,
Improving skin radiance,
Improving skin tone,
Skin soothing,
Preventing or reducing skin ageing, such as inflamm-aging or photo-aging,
Wrinkle smoothing,
Supporting skin regeneration,
Reducing signs of skin inflammation, such as itching, redness or swelling,
Reducing signs of skin fatigue.

In some embodiments, the invention relates to a method for improving skin complexion, the method comprising administering a composition comprising a compound of formula I onto a skin of an individual.

In some embodiments, the invention relates to a method for improving skin radiance, the method comprising administering a composition comprising a compound of formula I onto a skin of an individual.

In some embodiments, the invention relates to a method for improving skin tone, the method comprising administering a composition comprising a compound of formula I onto a skin of an individual.

In some embodiments, the invention relates to a method for skin soothing, the method comprising administering a composition comprising a compound of formula I onto a skin of an individual.

In some embodiments, the invention relates to a method for preventing or reducing skin ageing, such as inflamm-aging or photo-aging, the method comprising administering a composition comprising a compound of formula I onto a skin of an individual.

In some embodiments, the invention relates to a method for wrinkle smoothing, the method comprising administering a composition comprising a compound of formula I onto a skin of an individual.

In some embodiments, the invention relates to a method for supporting skin regeneration, the method comprising administering a composition comprising a compound of formula I onto a skin of an individual.

In some embodiments, the invention relates to a method for reducing signs of skin inflammation, such as itching, redness or swelling, the method comprising administering a composition comprising a compound of formula I onto a skin of an individual.

In some embodiments, the invention relates to a method for reducing signs of skin fatigue, the method comprising administering a composition comprising a compound of formula I onto a skin of an individual.

An improvement of skin complexion as used herein refers to the colour and texture of the skin, especially on the face.

Improving skin radiance as used herein refers to skin tone, luminosity, firmness, and discolouration (thin dark circles and sun damage).

Improving skin tone as used herein denotes normalizing natural hue of the skin, reducing redness and/or reducing pigmentation.

Skin soothing or skin calming, denotes herein protecting from skin irritation or reduction of irritated skin, e.g. mitigating, assuaging, or allaying pain from sunburn or skin dehydration.

Preventing or reducing skin ageing includes both chronological and premature aging, e.g the signs of ageing due to exposure of the skin to intrinsic and extrinsic factors, like decrease in production of essential molecules supporting the skin natural structure, loss of water, UV light damage, or exposure to chemical internal radicals generated in the body (due to unhealthy eating or life style, a disease, etc) or external pollutants hazardous for the skin.

Inflamm-aging is a chronic, low-grade inflammation that develops with advanced age, in the absence of overt infection, and may contribute to clinical manifestations of other age-related conditions. More precisely, inflamm-aging as used herein denotes skin inflammation as a biological response of a body tissue to both environmental challenges such as sun and chemical pollutants, and internal drivers such as diet, alcohol consumption and smoking, and other potentially harmful stimuli, including pathogens, bacteria, viruses, and other common irritants. Mechanistically, a low-grade, chronic and progressive prominence of pro-inflammatory cytokines over anti-inflammatory cytokines is typically occurring during inflamm-aging.

Photo-aging refers to the process of chronic sun exposure, including especially exposure to UV-A and UV-B radiation, leading to extrinsic skin aging. The structural and functional manifestations of cutaneous aging are typically premature and more severe in the photo-aged compared to the chronologically aged skin. Photo-aged skin tends to have a rather leathery appearance with deeper wrinkles and uneven pigmentation.

Wrinkle smoothing refers to herein, in particular, the face skin wrinkles, both fine surface lines and deeper furrows, i.e. lessen the appearance of wrinkled skin, smoothing the skin surface.

Supporting skin regeneration refers to the replacement of damaged skin cells with new fully functional cells. Skin regeneration typically happens in two possible ways, i.e. either by restoration or putting together what is broken, or by reconstruction or replacement and rebuilding what is torn down.

Skin fatigue as referred to herein is typically a result of long-term stress. This could e.g. be lifestyle-induced stress—such as lack of sleep or exercise, smoking, and unhealthy eating—but various environmental aggressors like pollution and UV radiation can also cause skin fatigue.

In general, cosmetic compositions of the invention support and enhance healthy look of the skin, especially of the face and neck skin.

The individual that receives cosmetic treatment according to the present invention may in some embodiments have a disease or a condition that affects the visual appearance of the skin.

Diseases that affect the visual appearance of the skin in the present context are typically not skin diseases, i.e. diseases directly affecting the skin, but other diseases that are not primarily skin-related, but only secondarily may also affect the visual appearance of the skin. Non-limiting examples include cardio-vascular diseases, metabolic diseases, gastro-intestinal diseases and, also mental disorders such as depression. Physiological conditions that may affect the visual appearance of the skin in the present context may be e.g. pregnancy or post-pregnancy. Physical trauma or surgical intervention is also considered in the context of the invention as physiological conditions that may affect visual appearance of skin of an individual that has experienced either of these conditions.

The individual that receives cosmetic treatment according to the present invention may in some embodiments have heredity of premature skin aging. For example, the individual may have a biological family in which early and strong wrinkle formation or sensitive skin is common.

The individual that receives cosmetic treatment according to the present invention may in some embodiments have been exposed to hazardous environmental factors. Hazardous environmental factors include, without being limited to, climatic conditions, such as UV light, or chemical pollutants, such as polycyclic aromatic pollutants (PAH), particulate matter PM2.5, blue light, ozone and cigarette smoke.

Topical compositions of the present invention can be structured or formulated into a variety of different forms. Non-limiting examples include emulsions (e.g., water-in-oil, water-in-oil-in-water, oil-in-water, silicone-in-water, water-in-silicone, oil-in-water-in-oil, oil-in-water-in-silicone emulsions), creams, lotions, solutions (both aqueous and hydroalcoholic), anhydrous bases (such as lipsticks and powders), gels, masks, peelings, and ointments. Variations and other structures will be apparent to the skilled artisan and are appropriate for use in the present invention.

In some embodiments, the topical composition may be formulated as an emulsion, an oil, a toner or a gel. In some embodiments, the topical composition may be formulated as an emulsion.

A topical composition of the present invention is typically intended for application on the face, on the body and/or on the hair. The topical composition preferably is for application on the face and/or on the body.

In preferred embodiments, the topical composition is formulated as an emulsion, an oil, a toner or a gel and for application on the face and/or on the body.

In some embodiments, the present invention relates to a composition that may be used for medical treatment of skin of an individual. In the context of the use of a composition for medical or pharmaceutical purposes, the topical composition may be referred to as a pharmaceutical composition or a pharmaceutical composition.

In some embodiments, the present invention relates to a topical pharmaceutical composition for use in treating of a skin disease in an individual, wherein the composition comprises a compound of formula I

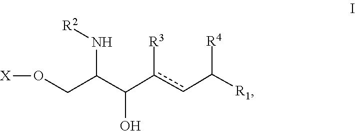

wherein
X is hydrogen, a glucosyl moiety or a lactosyl moiety,
the carbon-carbon bond noted [symbol] is a double or a single bond,
R¹ is an alkyl chain having 10-20 carbon atoms,
R² is hydrogen or a substituted or unsubstituted acyl having 16-34 carbon atoms,
R³ is hydrogen when the carbon-carbon bond noted [symbol] is a double or a single bond, or R³ is —OH when the carbon-carbon bond noted [symbol] is a single bond,
R⁴ is hydrogen when the carbon-carbon bond noted [symbol] is a double or a single bond, or R⁴ is hydrogen or —OH when the carbon-carbon bond noted [symbol] is a double bond.

In some embodiments, the compound of formula I of the topical pharmaceutical composition has a stereochemical configuration of (2S,3R,4E), and the carbon-carbon bond noted [symbol] is a double bond.

In some embodiments, the compound of formula I of the topical pharmaceutical composition has a stereochemical configuration of (2S,3S,4R) or (2S,3S), and the carbon-carbon bond noted [symbol] is a single bond.

In some embodiments, R¹ of the compound of formula I of the topical pharmaceutical composition is —C₁₂H₂₅ and/or R² is selected from hexadecanoyl, 2-hydroxyhexadecanoyl, octadecanoyl, 2-hydroxyoctadecanoyl, eicosanoyl, 2-hydroxyeicosanoyl, lignoceroyl, 2-hydroxytetracosanoyl, 30-(linoleyloxy)-triacontanoyl.

The term “skin disease” according to the invention is a disorder of structure or function in a human or animal that produces specific symptoms or that affects a specific location, i.e. skin, and is not simply a direct result of physical injury. Non-limiting examples of skin diseases include acne, rosacea, erythrocouperosis, psoriasis, xeroderma, ichthyosis, vascular disorders, diaper rash, atopic dermatitis, eczema, contact dermatitis, irritant dermatitis, allergic dermatitis, seborrheic dermatitis.

In some embodiments, the topical pharmaceutical composition further comprises an ingredient that is pharmaceutically active for treating a skin disease and is structurally different from a compound of formula I.

Non-limiting examples of such additional pharmaceutical active agents include anti-acne agents, agents used to treat rosacea, analgesics, anaesthetics, anorectals, antihistamines, anti-inflammatory agents including non-steroidal anti-inflammatory drugs, antibiotics, antifungals, antivirals, antimicrobials, anti-cancer actives, scabicides, pediculicides, antineoplastics, antiperspirants, antipruritics, antipsoriatic agents, antiseborrheic agents, biologically active proteins and peptides, burn treatment agents, cauterizing agents, depigmenting agents, depilatories, diaper rash treatment agents, enzymes, hair growth stimulants, hair growth retardants including DFMO and its salts and analogues, haemostatics, kerotolytics, canker sore treatment agents, cold sore treatment agents, dental and periodontal treatment agents, photosensitizing actives, skin protectant/barrier agents, steroids including hormones and corticosteroids, sunburn treatment agents, sunscreens, transdermal actives, nasal actives, vaginal actives, wart treatment agents, wound treatment agents, wound healing agents, etc.

Topical compositions of the invention may be advantageously administered onto skin of an individual for at least 7 days, such as for at least 14 days or for at least 21 days. For example, the topical composition is administered for at least 28 days. In practice, there is no time limitation for the administration period of a cosmetic compositions of the invention. For pharmaceutical composition of the invention, the administration period would be typically determined by a medical practitioner for each individual patient.

A topical composition (either cosmetic or pharmaceutical) may be administered one time or several times per day, such as 2-3 times per day. Typically, the topical composition is administered for consecutive days.

Topical compositions of the present invention typically include or are incorporated into different types of vehicles and carriers. In some embodiments, the topical composition of the present invention further comprises a topically acceptable carrier. The vehicle or carrier can be a pharmaceutically and/or dermatologically acceptable vehicle or carrier. Non-limiting examples of vehicles or carriers include water, glycerin, alcohol, oil, a silicon containing compound, a silicone compound, and wax. Variations and other appropriate vehicles will be apparent to the skilled artisan and are appropriate for use in the present invention. In certain aspects, the concentrations and combinations of the compounds, ingredients, and agents can be selected in such a way that the combinations are chemically compatible and do not form complexes which precipitate from the finished product.

In some embodiments, the topical composition may contain a C₆-C₁₀₀ ester base which functions as an acceptable carrier for the one or more compounds of formula I. Suitable examples of ester bases include:
(1) a C₇-C₆₀ neoalkanol ester. These esters may be formed from the reaction between C₁-C₂₂ alkanoic acid with neopentyl alcohol, neopentyl glycol, 2-butyl-2-ethyl-1,3-propanediol, 2,2,4-1rimethyl-1,3-pentane diol, trimethylol propane, pentaerythritol, di-trimethylol propane, di-pentaerythritol or pentaerythritol-trimethylol propane dimers. Examples include isostearyl neopentanoate, palmityl neopentanoate, tetraoctylpentaerythritol and diisopropyl neopentanoate. Amounts of this ester may suitably range from 1% to 90%, preferably from 20% to 75%, optimally from 30% to 50% by weight of the topical composition.
(2) a C₁₂-C₄₀ fatty glyceride ester alkoxylated with from 1 to 100 moles C₂-C₃ alkylene oxide per mole of glyceride. The C₁₂-C₄₀ fatty glyceride ester alkoxylate preferably includes 4 to 20 moles alkylene oxide per mole of glyceride. Preferred alkylene oxides are ethylene oxide and propylene oxide. Examples are PEG-6 caprylic/capric glyceride and PEG-8 caprylic/capric glyceride each of which are polyethylene glycol derivatives of a mixture of mono, di and triglycerides of caprylic and capric acids with a respective 6 and 8 moles of ethylene oxide. Amounts of this ester may suitably range from 1% to 90%, preferably from 5% to 50%, optimally from 10% to 20% by weight of the topical composition.
(3) a polyglycerol C₈-C₂₂ mono or di-fatty acid ester. Examples include polyglycerol-3 beeswax, polyglycerol-4 cocoate, polyglycerol-10 decalinoleate, polyglycerol-10 decaoleate, polyglycerol-7 decastearate, polyglycerol-2 diisostearate, polyglycerol-3 diisostearate, polyglycerol-7 diisostearate, polyglycerol-2 dioleate, polyglycerol-3 dioleate, polyglycerol-6 dioleate, polyglycerol-10 dioleate, polyglycerol-3 distearate, polyglycerol-6 distearate, polyglycerol-10 distearate, polyglycerol-10 heptaoleate, polyglycerol-10 heptastearate, polyglycerol-6 hexaoleate, polyglycerol-2 isostearate, polyglycerol-4 isostearate, polyglycerol-6 isostearate, polyglycerol-10 laurate, polyglycerol-10 myristate, polyglycerol-2 oleate, polyglycerol-3 oleate, polyglycerol-4 oleate, polyglycerol-6 oleate, polyglycerol-8 oleate, polyglycerol-10 oleate and combinations thereof. Most preferred is polyglycerol-6 dioleate which is a diester of oleic acid and a glycerin polymer containing an average of 6 glycerin units. Amounts of this ester may range from 1% to 50%, preferably from 5% to 25%, optimally from 10% to 20% by weight of the topical composition.
(4) a C₈-C₂₂ alkanol ester of a C₈-C₂₂ fatty acid. Non-limiting examples include cetyl octanoate, lauryl pentanoate, palmityl palmitoate, isostearyl decanoate, oleyl heptanoate and combinations thereof. Amounts of this material may range from 1% to 50%, preferably from 5% to 30%, optimally from 10% to 20% by weight of the topical composition.

The ester base may range from about 30% to 95%, preferably from 40% to 80%, optimally from 50% to 70% by weight of the topical composition.

The topical compositions may further contain a solubiliser in the form of a glycerol C₈-C₂₂ mono fatty acid ester. Non-limiting examples of solubilisers include glycerol monoisostearate, glycerol monobehenate and glycerol monopalmitate. The solubiliser may comprise about 0.1% to 20%, preferably from 0.5% to 10%, optimally from 1% to 5%, by mass of the topical composition.

The topical compositions may contain other carriers such as silicone oils. The silicone oils may be cyclic or linear polydimethylsiloxanes containing from 3 to 9, preferably from 4 to 5, silicon atoms, or polyalkyl siloxanes, polyalklyaryl siloxanes and polyether siloxane copolymers. Silicones may be present in amounts generally ranging from 1% to 50%, preferably from 2% to 25%, optimally between 10% and 20% by weight of the topical composition.

The topical compositions may also include additional ingredients typically found in cosmetic and skin care formulations such as cosmetic ingredients and pharmaceutical active ingredients.

Examples of further inclusions in topical compositions of the invention in different embodiments may include chalk, talc, fullers, earth, kaolin, starch, smectites clays, chemically modified magnesium aluminum silicate, organically modified montmorillonite clay, hydrated aluminium silicate, fumed silica, aluminum starch octenyl succinate. Also surfactants may be added including alkyl sulfates, such as Sodium Lauryl Sulfate, Sodium Laureth Sulfate, Sodium Coco-Sulfate and Sodium Trideceth Sulfate, betaines, such as cocobetaine, and carbohydrates, such as Lauryl Glucoside and Coco-Glucoside. Other ingredients are colouring agents, opacifiers and perfumes. Amounts of these adjunct materials may conveniently range anywhere from 0.001% up to 20% by weight of the composition.

The CTFA International Cosmetic Ingredient Dictionary and Handbook (2004 and 2008) describes a wide variety of non-limiting cosmetic ingredients that can be used in the context of the present invention. Examples of these ingredient classes include: fragrance agents (artificial and natural; e.g., gluconic acid, phenoxyethanol, and triethanolamine), dyes and colour ingredients (e.g., Blue 1, Blue 1 Lake, Red 40, titanium dioxide, D&C blue no. 4, D&C green no. 5, D&C orange no. 4, D&C red no. 17, D&C red no. 33, D&C violet no. 2, D&C yellow no. 10, and D&C yellow no. 11), flavouring agents/aroma agents (e.g., Stevia rebaudiana (sweetleaf) extract, and menthol), adsorbents, lubricants, solvents, moisturizers (including, e.g., emollients, humectants, film formers, occlusive agents, and agents that affect the natural moisturization mechanisms of the skin), water-repellents, UV absorbers and/or reflectors (physical and chemical absorbers such as para-aminobenzoic acid (“PABA”) and corresponding PABA derivatives, titanium dioxide, zinc oxide, etc.), essential oils, vitamins (e.g., A, B, C, D, E, and K), trace metals (e.g., zinc, calcium and selenium), anti-irritants (e.g., steroids and non-steroidal anti-inflammatories), botanical extracts (e.g., Aloe vera, chamomile, cucumber extract, Ginkgo biloba, ginseng, and rosemary), anti-microbial agents, antioxidants (e.g., BHT and tocopherol), chelating agents (e.g., disodium EDTA and tetrasodium EDTA), preservatives (e.g., methylparaben and propylparaben), pH adjusters (e.g., sodium hydroxide and citric acid), absorbents (e.g., aluminium starch octenylsuccinate, kaolin, corn starch, oat starch, cyclodextrin, talc, and zeolite), skin bleaching and lightening agents (e.g., hydroquinone and niacinamide lactate), humectants (e.g., sorbitol, urea, methyl gluceth-20, saccharide isomerate, and mannitol), exfoliants, waterproofing agents (e.g., magnesium/aluminium hydroxide stearate), skin conditioning agents (e.g., aloe extracts, allantoin, bisabolol, ceramides, dimethicone, hyaluronic acid, biosaccharide gum-1, ethylhexylglycerin, pentylene glycol, hydrogenated polydecene, octyldodecyl oleate, and dipotassium glycyrrhizate). Non-limiting examples of some of these ingredients are provided in the following subsections.

The topical compositions may also include fatty alcohols and fatty acids having from 10 to 36 carbon atoms. Especially preferred are such compounds as cetyl, myristyl, palmityl, isostearyl and stearyl alcohols and acids.

The topical compositions may further include α-hydroxy carboxylic acids. Suitable examples include: α-hydroxyethanoic acid, α-hydroxypropanoic acid, α-hydroxyhexanoic acid, α-hydroxyoctanoic acid, α-hydroxydecanoic acid, α-hydroxydodecanoic acid, α-hydroxytetradecanoic acid, α-hydroxyhexadecanoic acid, α-hydroxyoctadecanoic acid, α-hydroxyeicosanoic acid, α-hydroxydocosanoic acid, α-hydroxyhexacosanoic acid, and α-hydroxyoctacosanoic acid. The α-hydroxy carboxylic acid may be in the acid form or in salt form. Typical salts are the alkalimetal, ammonium and C₂-C₃₀ ammonium salts, for example the sodium, potassium, triethanolammonium and ammonium salts form. Typical salts are the alkalimetal, ammonium and C₂-C₃₀ ammonium salts, for example the sodium, potassium, triethanolammonium and ammonium salts. The α-hydroxy carboxylic acid will generally be present in a range from 0.001% to 20%, preferably from 0.01% to 15%, optimally from 0.5% to 10% by weight of the topical composition.

The topical composition may also include vitamins. Examples include sources or derivatives of vitamin C, such as ascorbyl palmitate, ascorbyl glucoside, magnesium ascorbyl phosphate, ascorbyl lysostearate, vitamin A palmitate (retinyl palmitate) and vitamin E linoleate (tocopheryl linoleate). Other esters of vitamin C, vitamin A and vitamin E may also be utilised.

The topical compositions may also include typical emulsifiers. The emulsifiers may be nonionic, anionic, cationic or amphoteric in nature. Examples of suitable emulsifiers include polyoxypropylene-polyoxyethylene ethers of cetyl alcohols and glycerol monoisostearate. The emulsifiers may suitably range from 0.5% to 30%, preferably from 1% to 15%, optimally from 3% to 8% by weight of the topical composition.

The topical compositions may also include thickeners. Examples include cross-linked polyacrylate materials and gums such as xanthan, carrageenan, gelatine, karaya, pectin and locust beans gum. The thickener may be present in amounts from about 0.1% to 20% by weight, preferably from 0.5% to 10% by weight, of the topical composition.

The topical compositions may also contain suitable preservatives. Suitable preservatives include alkyl esters of p-hydroxybenzoic acid, phenoxyethanol, hydantoin derivatives, proprionate salts, and a variety of quaternary ammonium compounds. For example, methyl paraben, propyl paraben, imidazolidinyl urea, sodium dehydroxyacetate and benzyl alcohol. Preservatives will usually be employed in amounts ranging from 0.1% to 2% by weight of the topical composition.

The topical composition may also include substances such as α-anisic acid, sodium anisate, levulinic acid, sodium levulinate, sodium hydroxide, caprylyl glycol, 1,2-hexanediol, pentylene glycol, glyceryl caprylate, glyceryl caprate, ethylhexylglycerin, Undecylenamidopropyl-trimonium Methosulfate, propylene glycol, phenylpropanol, phenethyl alcohol, methylpropanediol, Dipropylene Glycol, or a combination thereof.

Different aspects of the invention discussed above are described in the following selected, but not limiting, embodiments:
1. Cosmetic use of a composition for boosting skin cell energy metabolism in an individual, wherein the composition comprises one or more compounds of formula I

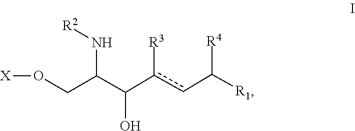

wherein
the carbon-carbon bond noted [symbol] is a double or a single bond,
R¹ is an alkyl chain having 10-20 carbon atoms,
R² is hydrogen or a substituted or unsubstituted acyl having 16-34 carbon atoms,
R³ is hydrogen when the carbon-carbon bond noted [symbol] is a double or a single bond, or
R³ is —OH when the carbon-carbon bond noted [symbol] is a single bond,
R⁴ is hydrogen when the carbon-carbon bond noted [symbol] is a double or a single bond, or
R⁴ is hydrogen or —OH when the carbon-carbon bond noted [symbol] is a double bond,
X is hydrogen, a glucosyl moiety or a lactosyl moiety.
2. Cosmetic use according to embodiment 1, wherein the composition is a topical composition.
3. Cosmetic use according to embodiment 1 or 2, wherein R¹ of the compound of formula I is —C₁₂H₂₅ and/or R² is selected from hexadecanoyl, 2-hydroxyhexadecanoyl, octadecanoyl, 2-hydroxyoctadecanoyl, eicosanoyl, 2-hydroxyeicosanoyl, lignoceroyl, 2-hydroxytetracosanoyl, 30-(linoleyloxy)-triacontanoyl.
4. Cosmetic use according to any one of embodiments 1 to 3, wherein the compound of formula I is present in an amount of about 0.0001% to about 10% wt/wt, such as from about 0.005% to about 5% wt/wt, or from about 0.05% to about 0.2% wt/wt.
5. Cosmetic use according to any one of embodiments 1 to 4, wherein the composition comprises
   a compound of formula I wherein X is glucosyl and R² is a substituted or unsubstituted acyl having 16-34 carbon atoms,
   a compound of formula I wherein X is lactosyl and R² is a substituted or unsubstituted acyl having 16-34 carbon atoms,
and/or
   a compound of formula I wherein X is hydrogen, R¹ is —C₁₂H₂₅ and R² is a substituted or unsubstituted acyl having 16-34 carbon atoms.
6. Cosmetic use according to any one of embodiments 1 to 5, wherein the composition further comprises coenzyme Q10, vitamin B3, vitamin C, creatine and/or resveratrol.
7. Cosmetic use according to any one of embodiments 1 to 6, having one or more of the following beneficial effects on the skin of an individual:
   Improving skin complexion,
   Improving skin radiance,
   Improving skin tone,
   Skin soothing,
   Preventing or reducing skin ageing, such as inflamm-aging or photoaging,
   Wrinkle smoothing,
   Supporting skin regeneration,
   Reducing signs of skin inflammation such as itching, redness or swelling,
   Reducing signs of skin fatigue.
8. Cosmetic use according to any one of embodiments 1 to 7, for activation of the respiratory chain and generation of ATP in skin cells.
9. Cosmetic use according to any one of embodiments 1 to 8, for activation of complex I.
10. Method for boosting skin cell energy metabolism, the method comprising topically administering a composition comprising a compound of formula I onto a skin of an individual

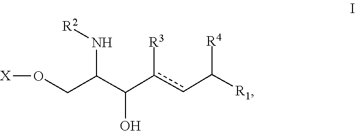

wherein
the carbon-carbon bond noted [symbol] is a double or a single bond,
R¹ is an alkyl chain having 10-20 carbon atoms,
R² is hydrogen or a substituted or unsubstituted acyl having 16-34 carbon atoms,
R³ is hydrogen when the carbon-carbon bond noted [symbol] is a double or a single bond, or
R³ is —OH when the carbon-carbon bond noted [symbol] is a single bond,
R⁴ is hydrogen when the carbon-carbon bond noted [symbol] is a double or a single bond, or
R⁴ is hydrogen or —OH when the carbon-carbon bond noted [symbol] is a double bond,
X is hydrogen, a glucosyl moiety or a lactosyl moiety.
11. Cosmetic use according to any one of embodiments 1 to 9 or method according to embodiment 10, wherein the individual is a human individual.
12. Cosmetic use or method according to any one of embodiments 1 to 11, wherein the individual has one or more of
   a disease that affects the visual appearance of the skin,
   heredity of premature skin aging, and/or
   been exposed to hazardous envinromental factors.
13. Cosmetic use or method according to embodiment 12, wherein the hazardous environmental factors include climatic conditions, such as UV light, or chemical pollutants, such as polycyclic aromatic pollutants (PAH), particulate matter PM2.5, blue light, ozone or cigarette smoke.
14. Cosmetic use or method according to any one of embodiments 1 to 13, wherein the composition is administered onto skin of an individual for at least 2 days, especially for at least 7 days or for at least 14 days, such as for 28 days.
15. Cosmetic topical composition for boosting skin cell energy metabolism in an individual, wherein the topical composition comprises one or more compounds of formula I

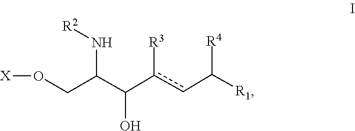

wherein
the carbon-carbon bond noted [symbol] is a double or a single bond,
R¹ is an alkyl chain having 10-20 carbon atoms,
R² is hydrogen or a substituted or unsubstituted acyl having 16-34 carbon atoms,
R³ is hydrogen when the carbon-carbon bond noted [symbol] is a double or a single bond, or
R³ is —OH when the carbon-carbon bond noted [symbol] is a single bond,
R⁴ is hydrogen when the carbon-carbon bond noted [symbol] is a double or a single bond, or
R⁴ is hydrogen or —OH when the carbon-carbon bond noted [symbol] is a double bond,
X is hydrogen, a glucosyl moiety or a lactosyl moiety,
wherein the composition has one or more of the following beneficial effects on the skin of an individual:
   Improving skin complexion,
   Improving skin radiance,
   Improving skin tone,
   Skin soothing,
   Preventing or reducing skin ageing, such as inflamm-aging or photoaging,
   Wrinkle smoothing,
   Supporting skin regeneration,
   Reducing signs of skin inflammation such as itching, redness or swelling,
   Reducing signs of skin fatigue.
16. Pharmaceutical composition comprising a compound of formula I, for use in treating a skin disease in an individual, such as psoriasis, keratoderma, dermatitis or atopic dermatitis.
17. Pharmaceutical composition according to embodiment 16, further comprising a compound that is
   structurally different from a compound of formula I and
   pharmaceutically active for treating of a skin disease.

### EXAMPLES

The examples described below are for illustration purposes only. The invention is not limited in scope to embodiments described in the examples.

### Example 1: Characterisation of Effects of Glucosyl Ceramide as Cosmetic Active Ingredient

Material and methods: Reconstructed Human Epidermis (RHE) cell culture was used, as this system mimics the in vivo 3D structure of epidermal tissue as well as the conditions and processes that occur in normal epidermis. RHE were cultured using the medium provided by the manufacturer until analysis. RHE were maintained in survival for 24 hours, before RHE were topically treated with lipid in a base emulsion for 24 h. 3 RHE were treated with base emulsion alone and 3 more remained untreated as a control batch (Ctrl). RHE were incubated under classical cell culture conditions (37° C., 5% CO2).

Sample preparation and LC-MS/MS analysis: Protein was extracted using the Pierce Total Protein Extraction Kit (Thermo Fisher) according to manufacturer's recommendations. The protein was mixed with Laemmli buffer and heated to 37° C. for 30 min. Protein concentration was determined by the BCA method and standardized for all samples. The samples were separated by SDS-PAGE and digested with trypsin overnight. The peptides generated were acidified and separated. The mass spectra were queried using Proteome Discoverer (version 2.5). The resulting MS/MS data were queried against the Homo sapiens proteome UP000005640 (20371 reviewed entries). Results were filtered based on unique peptides>2, and global peptide scores. Analyses were performed using Proteome Discoverer 2.5 to check for overall sample quality.

Bioinformatics and statistical analysis: Proteomic analysis returned a mean total of 34961 high-quality peptides corresponding to a mean 2422 identifiable and quantifiable RHE proteins among conditions. Proteins with a p-value<0.05 in at least one of the concentrations tested for a lipid were considered significant and used for pairwise comparisons. Relevant proteins were classified by their biological processes and associated pathways using the publicly available gene ontology (GO) database provided by the Gene Ontology Consortium and Reactome. Interactome analysis was performed using Cytoscape combined with STRING.

### Example 2: Characterisation of Effect of Glucosyl Ceramide on Skin Cell Energy Using Skin explantsThe Aim of the Study is to Evaluate the Effect of Glucosyl Ceramide on Cell Energy Using Living Human Skin Explants. The Activity is Evaluated by a Biochemical Quantification of Complex I

Complex I, found in mitochondria, is the first of five mitochondrial complexes that carry out a multi-step process called oxidative phosphorylation, through which cells derive much of their energy. The analysis is based on the determination of Complex I activity in skin explants.

### 2.1 Explant Preparation:

Human skin explants of an average diameter of 11 mm (±1 mm) were prepared on an abdoplasty coming from a 57-year-old Caucasian woman with a II phototype according to Fitzpatrick skin colour classification. The explants were kept in survival in BEM culture medium (BIO-EC's Explants Medium) at 37° C. in a humid, 5%-CO2 atmosphere.

TABLE 1
Explants distribution:

| | | Number of | |
|---|---|---|---|
| Batch | Conditions | explants | Sampling |
| T0 | Tissue control | 3 | Day 0 |
| T | Non-treated control | 4 | Day 6 |
| E | Basic emulsion from Cosmacon GmbH | 4 | Day 6 |
| | (composition in Table 2) | | |
| P | Basic emulsion from Cosmacon GmbH | 4 | Day 6 |
| | with 0.05% GlcCNS18 | | |

TABLE 2
Composition of basic emulsion from Cosmacon GmbH:

| Phase | Raw ingredient | INCI |
|---|---|---|
| Oily | Lanette 18 | Stearyl alcohol |
| phase | Emulgade PL 68/50 | Cetearyl Alcohol, Cetearyl |
| | | Glucoside |
| | Eutanol G | Octyldodecanol |
| Water | Water, demin | Aqua |
| phase | Glycerin 99.5% | Glycerin, Aqua |
| Thickening | Microcare Emollient PTG | Pentylene Glycol |
| phase | Keltrol CG -SFT V | Xanthan Gum |

### 2.2 Product Application:

The tested products E and P were applied topically at a rate of 2 μL per 1 cm² explant (2 mg/cm²), and spread using a small spatula on day 0 (D0), D1, D2 and D5. The control explants T did not receive any treatment except the renewal of culture medium. The culture medium was half renewed (1 ml per well) on D1, D2 and completely renewed (2 mL per well) on D5.

### 2.3 Sampling:

On D0, the 3 explants from the batch TO were collected and cut in three parts. One third was fixed in buffered formalin solution, one third was frozen at −80° C. for histological analysis and one third was frozen at −80° C. for dosages of ATP Complex I (NADH Dehydrogenase) realized by Integracell. On D6, 4 explants from the concerned batches were collected and treated in the same way than in DO. The culture medium BEM of all batches was collected on D6 (2 mL per explant) and stored at −20° C. for dosages.

### 2.4 Histological Processing and Cell Viability:

After fixation for 24 hours in buffered formalin, the samples were dehydrated and impregnated in paraffin using a Leica PEARL dehydration automat. The samples were embedded using a Leica EG 1160 embedding station. 5-μm-thick sections were made using a Leica RM 2125 Minot-type microtome, and the sections were mounted on Superfrost® histological glass slides. The microscopical observations were realized using a Leica DMLB, an Olympus BX43 or BX63 microscope. Pictures were digitized with a numeric DP72 or DP74 Olympus camera with cellSens storing software (Olympus).

The cell viability of the epidermal and dermal structures was assessed by microscopical observation of formalin-fixed paraffin-embedded (FFPE) skin sections after Masson's trichrome staining, Goldner variant. The staining was assessed by microscopical observation.

### 2.5 Biochemical and Enzymatic Quantification Assays:

The frozen explants (one third) were grinded in phosphate buffer pH=7.8 using an ultra homogenizer (Turax) and a cell disruption was then processed using Ultrasonic disruptor (Bransonic). After centrifugation at +4000 tr/min at 18° C., the supernatants of all explants were harvested for the dosages of total proteins, citrate synthase (CS) activity and Complex I (NADH Dehydrogenase) activity by Integracell.

### 2.5.1 Dosage of Total Proteins:

The total proteins were quantified by automate Bradford technique using Bradford reagent. The concentration of total proteins of each explant was expressed in g/L.

### 2.5.2 Citrate Synthase Activity Assay:

Citrate synthase is commonly used as a quantitative enzyme marker for the presence of intact mitochondria. Citrate synthase activity assay was realized with an enzymatic method. The enzyme catalyses the reaction between acetyl coenzymeA (acetyl CoA) and oxaloacetic acid to form citric acid. The reaction capacity of the citrate synthase was followed by measuring the formation of the —SH group released from CoA-SH by use of the reactive Ellman reagent (5,5′-dithiobis [2-nitrobenzoic], DTNB) and monitoring the absorbance at 415 nm.

Using this assay, citrate synthase activity was measured on day 0 and day 6. The citrate synthase activity was measured in international unit per grams of total proteins (U/g prot).

### 2.5.3 Complex I Activity Assay:

Complex I (CP1) activity assay was realized with an enzymatic method. This assay is based on the transformation of NADH by NADH Dehydrogenase, followed by monitoring the absorbance at 340 nm. Using this assay, complex I activity was measured on day 0 and day 6.

The complex I activity was expressed in unit of complex per unit of citrate synthase (U/CS). It represents the complex I activity in the mitochondria. The complex I activity was also expressed in unit of complex grams of total proteins (U/g prot). It represents the global complex I activity in the cell.

### 2.6 Results:

### 2.6.1 Cell Viability:

On D0 and D6, cell viability is good in the epidermis and in the dermis. Products E and P induce no modification on the cell viability.

### 2.6.2 Dosage of Complex I:

The activity of complex I (NADH Dehydrogenase) in the explants (expressed in U/g prot) represents the global complex I activity in the cell, for all batches shown here below:

TABLE 3
Complex I activity in explants (U/g proteins):

| | T0 | T6 | E6 | P6 |
|---|---|---|---|---|
| Mean | 28.0 | 39.7 | 42.7 | 49.2 |
| Standard Deviation (SD) | 5.2 | 4.3 | 3.6 | 4.4 |

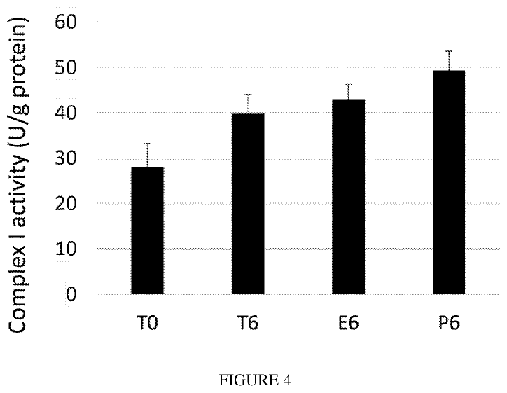

Product E induces a non-significant increase of 8% on complex I activity compared to T6. Product P induces a significant increase of 24% on complex I activity compared to T6. Results are also presented as graphs in FIG. 4.

The above-described embodiments are combinable. The following dependent claims set out particular embodiments of the invention.

1. Cosmetic use of a composition for boosting skin cell energy metabolism in an individual, wherein the composition comprises one or more compounds of formula I 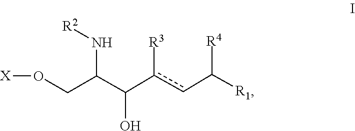
wherein
the carbon-carbon bond noted [symbol] is a double or a single bond,
R¹ is an alkyl chain having 10-20 carbon atoms,
R² is hydrogen or a substituted or unsubstituted acyl having 16-34 carbon atoms,
R³ is hydrogen when the carbon-carbon bond noted [symbol] is a double or a single bond, or
R³ is —OH when the carbon-carbon bond noted [symbol] is a single bond,
R⁴ is hydrogen when the carbon-carbon bond noted [symbol] is a double or a single bond, or
R⁴ is hydrogen or —OH when the carbon-carbon bond noted [symbol] is a double bond,
X is hydrogen, a glucosyl moiety or a lactosyl moiety.

2. The cosmetic use according to claim 1, wherein the composition is a topical composition.

3. The cosmetic use according to claim 1, wherein R¹ of the compound of formula I is —C₁₂H₂₅ and/or R² is selected from hexadecanoyl, 2-hydroxyhexadecanoyl, octadecanoyl, 2-hydroxyoctadecanoyl, eicosanoyl, 2-hydroxyeicosanoyl, lignoceroyl, 2-hydroxytetracosanoyl, and 30-(linoleyloxy)-triacontanoyl.

4. The cosmetic use according to claim 1, wherein the compound of formula I is present in an amount of about 0.0001% to about 10% wt/wt.

5. The cosmetic use according to claim 1, wherein the composition comprises
(a) a compound of formula I wherein X is glucosyl and R² is a substituted or unsubstituted acyl having 16-34 carbon atoms,
(b) a compound of formula I wherein X is lactosyl and R² is a substituted or unsubstituted acyl having 16-34 carbon atoms,
and/or
(c) a compound of formula I wherein X is hydrogen, R¹ is —C₁₂H₂₅, and R² is a substituted or unsubstituted acyl having 16-34 carbon atoms.

6. The cosmetic use according to claim 1, wherein the composition further comprises an enzyme cofactor, a vitamin, an anti-oxidant, and/or an agent facilitating ATP regeneration.

7. The cosmetic use according to claim 1, having one or more of the following beneficial effects on the skin of an individual:
(a) Improving skin complexion,
(b) Improving skin radiance,
(c) Improving skin tone,
(d) Skin soothing,
(e) Preventing or reducing skin ageing, inflamm-aging, or photoaging,
(f) Wrinkle smoothing,
(g) Supporting skin regeneration,
(h) Reducing signs of skin inflammation, and
(j) Reducing signs of skin fatigue.

8. The cosmetic use according to claim 1, for activation of the respiratory chain and/or generation of ATP in skin cells.

9. The cosmetic use according to claim 1, for activation of complex I.

10. Method for boosting skin cell energy metabolism, the method comprising topically administering a composition comprising a compound of formula I onto a skin of an individual 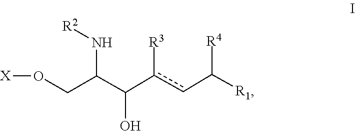
wherein
the carbon-carbon bond noted [symbol] is a double or a single bond,
R¹ is an alkyl chain having 10-20 carbon atoms,
R² is hydrogen or a substituted or unsubstituted acyl having 16-34 carbon atoms,
R³ is hydrogen when the carbon-carbon bond noted [symbol] is a double or a single bond, or
R³ is —OH when the carbon-carbon bond noted [symbol] is a single bond,
R⁴ is hydrogen when the carbon-carbon bond noted [symbol] is a double or a single bond, or
R⁴ is hydrogen or —OH when the carbon-carbon bond noted [symbol] is a double bond,
X is hydrogen, a glucosyl moiety or a lactosyl moiety.

11. The method according to claim 10, wherein the individual is a human individual.

12. The method according to claim 11, wherein the individual has or had one or more of:
(a) disease or a physiological condition that affects the visual appearance of the skin,
(b) heredity of premature skin aging,
(c) exposure to hazardous environmental factors.

13. The method according to claim 12, wherein the hazardous environmental factors comprise at least one of UV light, a chemical pollutant, a polycyclic aromatic pollutant (PAH), particulate matter PM2.5, blue light, ozone, and cigarette smoke.

14. The method according to claim 13, wherein the composition is administered onto the skin of an individual for at least 2 days.

15. Cosmetic topical composition for boosting skin cell energy metabolism in an individual, wherein the topical composition comprises one or more compounds of formula I 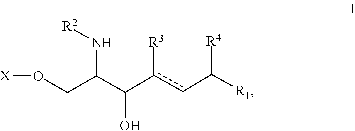
wherein
the carbon-carbon bond noted [symbol] is a double or a single bond,
R¹ is an alkyl chain having 10-20 carbon atoms,
R² is hydrogen or a substituted or unsubstituted acyl having 16-34 carbon atoms,
R³ is hydrogen when the carbon-carbon bond noted [symbol] is a double or a single bond, or
R³ is —OH when the carbon-carbon bond noted [symbol] is a single bond,
R⁴ is hydrogen when the carbon-carbon bond noted [symbol] is a double or a single bond, or
R⁴ is hydrogen or —OH when the carbon-carbon bond noted [symbol] is a double bond,
X is hydrogen, a glucosyl moiety or a lactosyl moiety,
wherein the composition has one or more of the following beneficial effects on the skin of an individual:
(a) Improving skin complexion,
(b) Improving skin radiance,
(c) Improving skin tone,
(d) Skin soothing,
(e) Preventing or reducing skin ageing, inflamm-aging, or photoaging,
(f) Wrinkle smoothing,
(g) Supporting skin regeneration,
(h) Reducing signs of skin inflammation, and
(i) Reducing signs of skin fatigue.

16. (canceled)

17. (canceled)

18. The cosmetic use according to claim 4, wherein the compound of formula I is present in an amount of about 0.005% to about 5% wt/wt or about 0.05% to about 0.2% wt/wt.

19. The cosmetic use according to claim 6, wherein the composition comprises one or more of the following:
(a) coenzyme Q10,
(b) vitamin B3,
(c) vitamin C,
(d) astaxanthin,
(e) resveratrol, and
(f) creatine.

20. The cosmetic use according to claim 7, wherein reducing signs of inflammation comprises reducing one or more of itching, redness, and swelling.

21. The method according to claim 12, wherein the disease or physiological condition that affects the visual appearance of the skin comprises physical trauma or psychological trauma.

22. The method according to claim 14, wherein the composition is administered onto the skin of the individual for at least 7 days, at least 14 days, or at least 28 days.
